(19) United States
(12) Patent Application Publication
(10) Pub. No.: US 2009/0062807 A1
(43) Pub. Date: Mar. 5, 2009

(54) Device and method for placement of interbody device

(75) Inventor: John K. Song, Chicago, IL (US)

Correspondence Address: JOHN K. SONG, 474 NORTH LAKE SHORE DR., APT. # 4508, CHICAGO, IL, 60611-6465, US

(73) Assignee: Vermillion Technologies, LLC, Chicago, IL (US)

(21) Appl. No.: 11/895,652

(22) Filed: Aug. 27, 2007

(51) Int. Cl. A61B 17/58; A61B 1/00; A61F 5/00

(52) U.S. Cl. 606/87; 600/101; 606/79

(57) ABSTRACT

A method for performing percutaneous interbody preparation and placement of an interbody device is disclosed. The method includes the steps of inserting a guide mechanism into the disc space from a posterolateral orientation and then working over the guide mechanism with cannulated, self-distracting tools and implants to prepare and place the desired implant. Means for performing the percutaneous interbody preparation and implant placement procedure are also disclosed.

### FIELD OF THE INVENTION

The present invention relates to a device for percutaneously preparing an interbody space and placing a device within the interbody space of the spine.

### BACKGROUND OF THE INVENTION

Spinal fusion surgery is the joining together of vertebrae of the spine. The underlying concept of fusion surgery implants is to maintain the relative position of the vertebral bodies with respect to eachother while bone graft placed between the vertebrae has an opportunity to heal and mature. These devices employ different strategies and philosophies, such as devices which utilize the pedicles, and devices which are placed in the disc space. The latter devices and techniques associated with these devices are known as “interbody fusion” devices. While no single technique has been universally accepted as the most optimum method, there is growing evidence that interbody fusion may be the preferred method.

Initially, all interbody fusion procedures were accomplished using a posterior approach. The procedure typically begins with a laminectomy, followed by removal of the intervertebral disc, and then in most cases the height of the disc space is increased and packed with pieces of bone. The goal is for the inserted bone pieces to fuse together with the vertebrae above and below that disc space, forming a bridge of bone and therefore eliminating motion at that spinal level.

More recently, interbody fusion performed by an anterior procedure has gained popularity. Anterior fusion instruments typically provide for a retroperitoneal or transperitoneal approach to the lumbar disc. Some or all of the disc is removed, and either bone or a metallic device is placed into the disc space. These devices also typically provide a means for distracting the disc space, i.e. making the space between the discs wider. Presently, this aspect of lumbar interbody fusion procedures are considered to be an important step in the procedure because of its effects on the neural foramina, or areas from which the nerve roots exit through the vertebra. It is generally accepted that enlarging the disc space consequently enlarges the neural foramina, thus decompressing the exiting nerve roots.

Due to presently available equipment, current anterior interbody fusion surgeries are disadvantageously invasive, requiring large incisions and manipulation of both tissue and organs. While attempts have been made to perform anterior interbody fusions laparoscopically, these procedures are often complicated and are typically performed under general anesthesia.

Therefore, a need exists for an interbody fusion method which reduces trauma to the patient; consequently reducing recovery time. Implant and instruments for percutaneous anterior interbody fusion; enabling the surgeon to distract the disc space to restore disc height, maintain the distraction, and promote the growth of interbody bone graft, would satisfy this heretofore unaddressed need.

Most commonly, surgery over a wire or through a tube has been used for placement of devices into bones of the spine. However, the technique of the current invention has not been applied for disc space preparation and device placement.

Numerous prior art methods and devices exist in this arena. Some describe utilization of wires or tubes for percutaneous placement of interbody devices—however, most do not employ direct entry into the interbody space itself with percutaneous instruments. U.S. Pat. No. 6,666,891 to Boehm et al describes a system whereby a guide wire is placed into the discspace and a sequence of serial dilators is used to create a working channel through which disc space preparation is performed. An expandable interbody fusion device is then placed and the guide tube removed.

The current invention describes a method and means of placing an interbody device percutaneously into the space between vertebrae with a substantially posterior, posterolateral, or lateral approach. The device itself may comprise a device for purposes of fusion or for other purposes such as nucleus or disc replacement or placement of biologic substances. One embodiment of the current invention differs significantly from that of Boehm et al in that the need for a guide wire and an expandable interbody device are eliminated. The working tube of the present invention allows preparation of the endplates of the vertebra through windows in the side. This allows more stable placement of a longer tube into the disc space while maintaining access to the endplates. In yet another embodiment, the working tube with side openings also becomes the delivery device and implant. The present invention includes methods and devices for both fusion and non-fusion spinal surgeries.

U.S. Patent Documents

| | | |
|---|---|---|
| 3811449 | May 1974 | Gravlee et al. |
| 5015247 | May 1991 | Michelson |
| 5522899 | June 1996 | Michelson |
| 5609635 | March 1997 | Michelson |
| 5665122 | September 1997 | Kambin |
| 5782832 | July 1998 | Larsen et al. |
| 6083225 | July 2000 | Winslow et al. |
| 6113602 | September 2000 | Sand |
| 6126689 | October 2000 | Brett |
| 6129763 | October 2000 | Chauvin et al. |
| 6395034 | May 2002 | Suddaby |
| 6419705 | July 2002 | Erickson |
| 6527734 | March 2003 | Cragg et al. |
| 6666891 | December 2003 | Boehm et al. |

Foreign Patent Documents

| | | |
|---|---|---|
| 44 16 605 | November, 1994 | DE |
| 98 10832 | August, 1998 | FR |
| WO 96/27321 | September, 1996 | WO |
| WO 00/35388 | June, 2000 | WO |
| WO 00/49977 | August, 2000 | WO |

### SUMMARY OF THE INVENTION

While the invention has been shown and described with reference to certain preferred embodiments, it will be understood by those skilled in the art that various changes and modifications in form and detail may be made therein without departing from the spirit and scope of the invention, as defined by the appended claims.

It is an object of the present invention to present a simplified, minimally invasive, percutaneous surgical procedure for placement of an interbody device.

It is also an object of the present invention to present a method of percutaneous interbody fusion surgery where the interbody device is implanted through small incisions and a minimum of incisions, thereby reducing trauma to the patient.

It is also an object of the present invention to present a means of percutaneously preparing an interbody space for accepting an implant.

It is also an object of the present invention to eliminate the need for serial dilation.

It is also an object of the present invention to eliminate the need for placement of a working tube.

It is also an object of the present invention to provide a device which can be inserted over a guide wire and which distracts the disc space.

It is also an object of the invention to provide examples of several instruments for preparation of the interbody space for receiving said interbody device. The instruments and device incorporate self-distracting features to facilitate placement and distraction of the interbody space.

It is also an object to present instruments which have tapered or rounded features to minimize entanglement of soft tissue when withdrawing the instruments from the body.

It is also an object of the present invention to provide a kit for performing a minimally invasive percutaneous interbody procedure.

A method for placing a percutaneous interbody device is also provided, in which a guide mechanism (e.g. a wire) is first placed into the disc space, possible guided by a hollow needle with an internal diameter larger than the guide mechanism, such as a bone marrow biopsy or “Jamshidi” needle. Placement and use of all instruments may be guided by fluoroscopy or other intraoperative navigation tool or tools.

Once the guide wire is placed, a wide variety of instrument constructions may be utilized which mate with and are guided by the guide mechanism. The following procedure is given as an example and is therefore not intended to limit the scope of the invention:
1) A guide mechanism is surgically placed into an interbody space of an intervertebral disc.
2) An awl or other device with a pointed leading edge is guided by the guide mechanism to the intervertebral disc and is used to create an opening in the disc annulus.
3) A reamer or drill is guided to the disc space by the guide mechanism and is used to break up the disc and possibly remove a portion of the vertebral endplate. The reamer or drill has a tapered leading edge to distract the disc space by a wedge action. The distraction will be maintained while the instrument is being used.
4) Other instruments, including hollow tubes or fluted shafts, may be used in the same way as the reamer or drill to remove disc material. These instruments would also utilize a tapered leading edge to facilitate entry into the disc space and to maintain distraction.
5) A trial sizer may be guided to the disc space by the guide mechanism to determine the correct implant size to use. The sizer may be a cylindrical or square block with tapered leading edges or may be a “twist” type sizer which is rotated to distract the vertebral bodies.
6) Once the disc space is adequately prepared, the interbody implant itself may be guided into the disc space with the guide mechanism. The interbody implant would incorporate a tapered leading edge to distract the interbody space. Unilateral or bilateral implantation may be performed by this method. If the interbody implant is to be used to fuse the interbody space, it may incorporate bone graft material which may be added to the implant before or after placement in the interbody space.
7) The guide mechanism is removed.

A kit for performing percutaneous interbody preparation is also provided which includes the guide mechanism, means for placing the guide mechanism, instruments required for performing disc removal and disc space preparation, and possibly an interbody implant with an insertion handle.

All instruments may also have a tapered or rounded trailing edge to facilitate removal of the instrument from the body and to prevent damage to soft tissues.

The device of the present invention utilizes a method of preparing a disc space for accepting an implant whereby all instruments and implants are placed over the guide mechanism and all instruments and implants have a tapered or similar leading feature. While the preferred embodiment of the guide mechanism is a wire, other embodiments could include any configuration whereby the subsequent instruments and implants substantially encompass and surround the guide mechanism even if only partially.

The preferred embodiment of the guide mechanism is a wire which is long enough to enter the interbody space and protrude from the skin incision. Alternative embodiments include: solid or hollow rods of various cross-section, or the instruments and implants may not circumscribe the guide mechanism but may have a feature such as a slot along which the instruments and implants are passed. For example, the guide mechanism may be a square bar having one or more longitudinal slots or grooves which cooperate with a feature on the instruments and implants.

All instruments and implants could potentially incorporate means for surgical tracking or intra-operative nerve monitoring to minimize injury to nervous structures.

### BRIEF DESCRIPTION OF THE DRAWINGS

FIG. 1A is an anterior view of two adjacent vertebrae showing placement of the guide wire.

FIG. 1B is a top view of two adjacent vertebrae showing placement of the guide wire.

FIG. 1C is an oblique view of two adjacent vertebrae showing placement of the guide wire.

FIG. 2A is an oblique view showing a cannulated awl being passed over the guide wire and into the intervertebral disc space.

FIG. 2B is an isolated view showing a cannulated awl being passed over the guide wire

FIG. 3 shows an instrument for removing intervertebral disc material and preparing endplates.

FIG. 4 shows an instrument for removing intervertebral disc material and preparing endplates.

FIG. 5 shows an instrument for removing intervertebral disc material and preparing endplates.

FIG. 6 shows an instrument for preparing a vertebra for fusion.

FIG. 7A shows a fusion implant and a handle for insertion into a disc space.

FIG. 7B shows a fusion implant attached to a handle for insertion into a disc space.

FIG. 8 shows a fusion implant being inserted over a guide wire into the disc space.

### DETAILED DESCRIPTION OF THE DRAWINGS

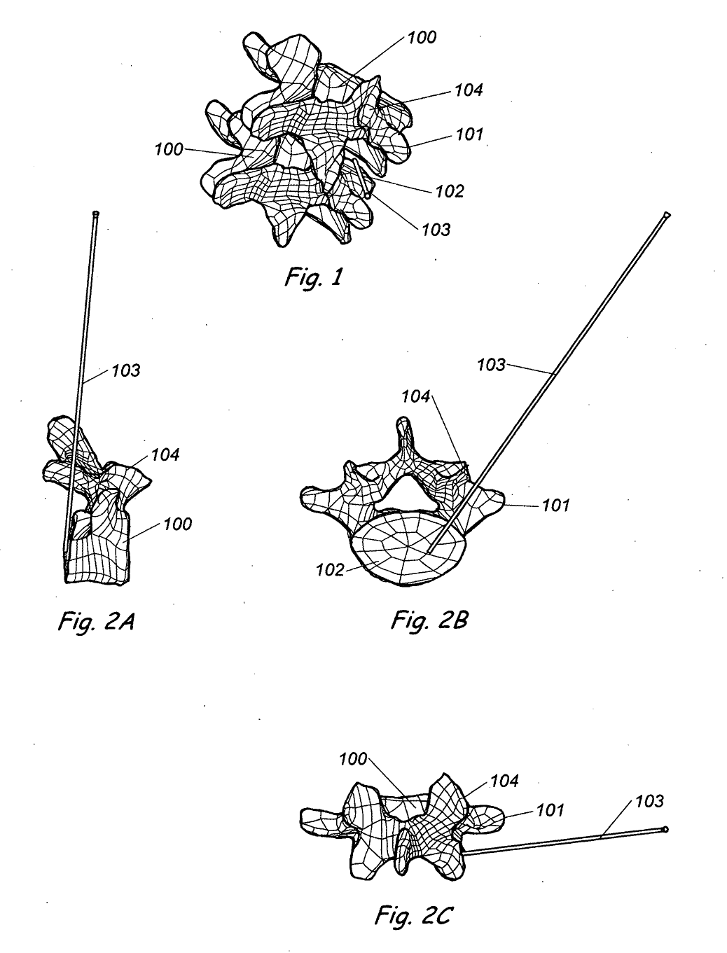

FIGS. 1A to 1C show the initial step of the percutaneous fusion method, in which a wire 50 is inserted through the patient's skin and into the disc space 15. FIG. 1A is an anterior view, FIG. 1B is a caudal view, and FIG. 1C is an oblique view. Typically, wire 50 would be guided into position with the aid of a fluoroscope.

FIGS. 2A and 2B show the second step of the percutaneous fusion method, in which a cannulated awl 100 is guided over the wire placed in the initial step and into the disc. The sharp leading edge 102 of cannulated awl 100 is used to perforate the annulus of the intervertebral disc. A handle 108 facilitates manipulation of awl 100. Wire 50 guides the insertion of cannulated awl 100.

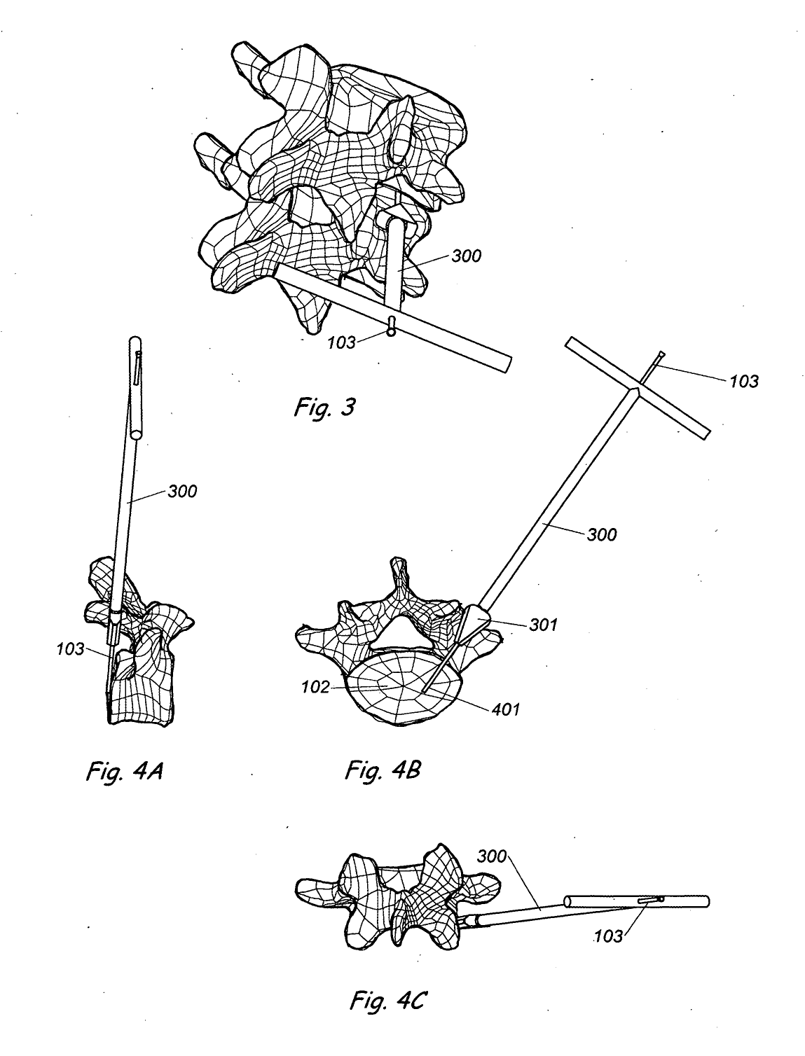

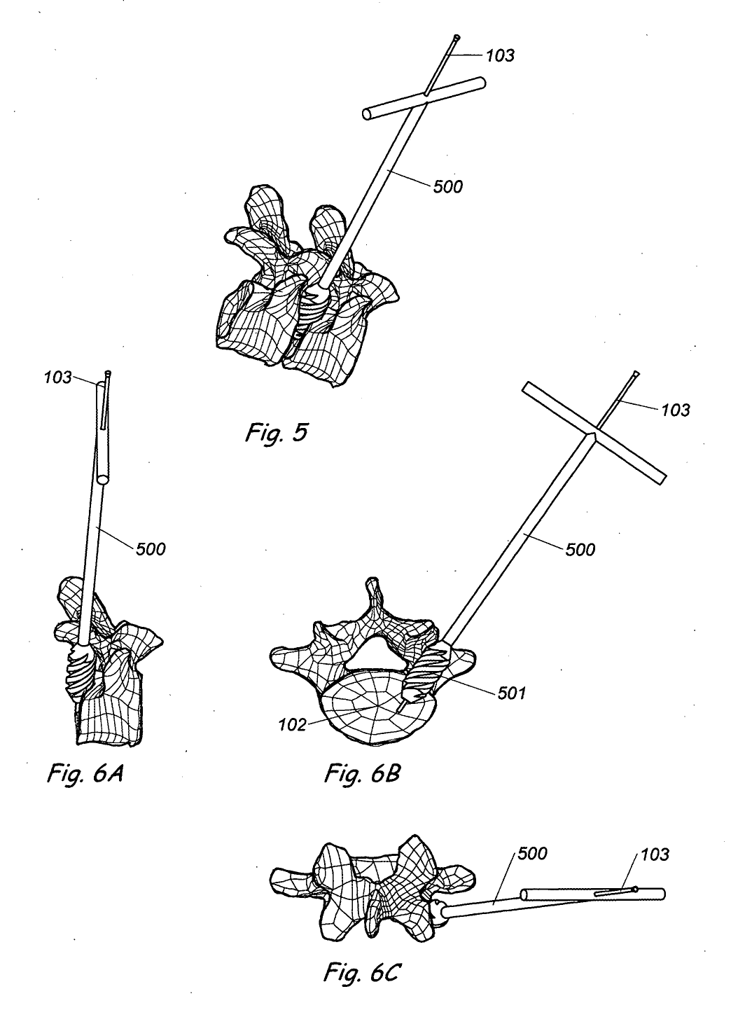

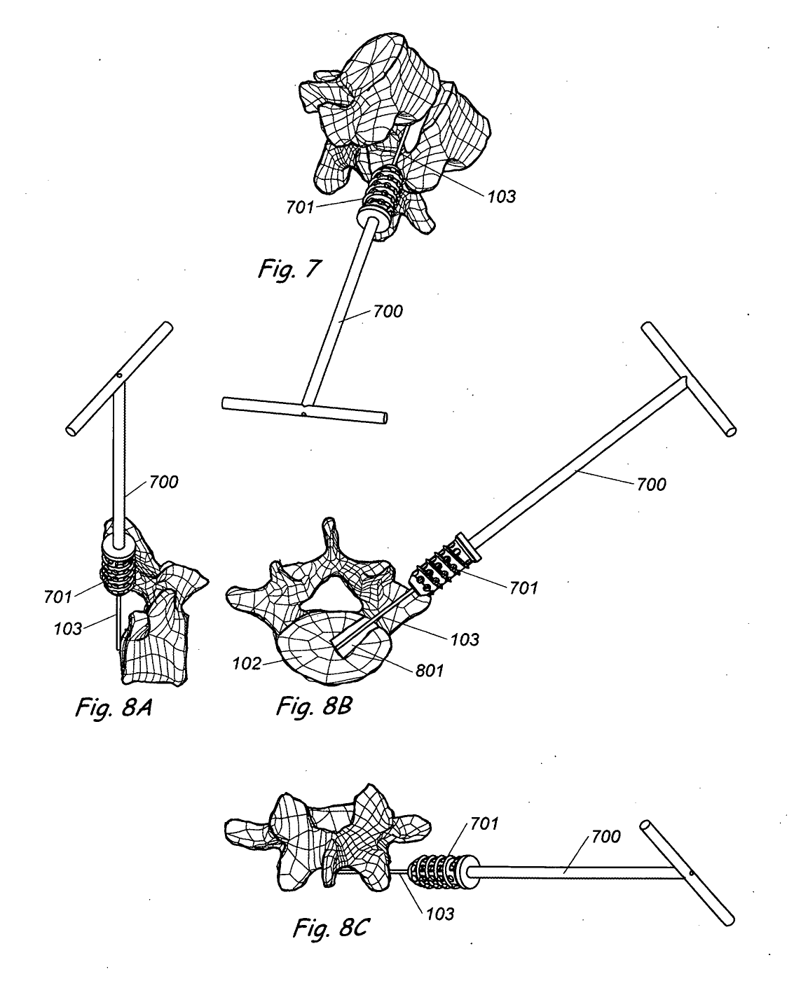

FIGS. 3 to 6 show various instruments which could be useful for performing the percutaneous fusion methos. Their operation is similar to the operation of cannulated awl 100 in that they are all passed over wire 50 and therefore guided into disc space 15. FIG. 3 shows a cannulated auger 200 which consists of a shaft 204 with a handle 212, and a cutting head 202 with a tapered nose 206, a helical cutting groove 208, and a guide hole 210 which passes completely or partially through cannulated auger 200. Tapered nose 206 aids in insertion of cannulated auger 200 by distracting disc space 15 as it is tapped in. Helical cutting groove 208 creates a cutting edge on cannulated auger 200 and also aids in clearing the cut material out of the disc space 15. The purpose of this instrument is to remove intervertebral disc material, vertebral endplate material, and/or vertebral bone. FIG. 4 shows a cannulated scraper 300 which consists of a shaft 304, with a handle 312, and a cutting head 302 with a tapered nose 306, a helical cutting thread 308, and a guide hole 310 which passes completely or partially through cannulated auger 300. Tapered nose 306 aids in insertion of cannulated auger 300 by distracting disc space 15 as it is tapped in. Helical cutting thread 308 creates a scraping edge on cannulated auger 200 and also aids in clearing the cut material out of the disc space 15. The purpose of this instrument is to remove intervertebral disc material, vertebral endplate material, and/or vertebral bone. FIG. 5 shows a cannulated rasp 400 which consists of a shaft 404, with a handle 412, and a cutting head 402 with a tapered nose 406, cutting holes 408, and a guide hole 410 which passes completely or partially through cannulated rasp 400. Tapered nose 406 aids in insertion of cannulated rasp 400 by distracting disc space 15 as it is tapped in. Cutting holes 408 create cutting edges on cannulated rasp 400 and also trap the cut material. The purpose of this instrument is to remove intervertebral disc material, vertebral endplate material, and/or vertebral bone.

What is claimed is:

1. A method of percutaneously preparing an interbody space for placement of a percutaneous interbody device comprising the following steps:
a. Making a skin incision overlying an interbody space, and;
b. Inserting a guide mechanism into the interbody space between a first and second vertebra, and;
c. Utilizing one or more instruments with a self-distracting leading edge over the guide mechanism to prepare the interbody space;
d. Placement of at least one interbody implant utilizing said guide mechanism.

2. The method of claim 1 wherein said guide mechanism is placed through a trocar.

3. The method of claim 1, wherein said guide mechanism is inserted from an orientation which is substantially posterior to the spine of the patient.

4. The method of claim 1, wherein said guide mechanism is inserted from an orientation which is substantially posterior-lateral to the spine of the patient.

5. The method of claim 1, wherein said guide mechanism is inserted from an orientation which is substantially lateral to the spine of the patient.

6. The method of claim 1, wherein insertion of said guide mechanism, said cutting instrument, or said interbody implant utilizes a monitoring tool such as a fluoroscope.

7. The method of claim 1 wherein said method can be applied unilaterally or bilaterally.

8. The method of claim 1 wherein said at least one cutting instrument, said at least one sizing instrument or said at least one interbody implant has a rounded or tapered leading edge.

9. The method of claim 1 wherein said at least one cutting instrument, said at least one sizing instrument or said at least one interbody implant has a rounded or tapered trailing edge.

10. The method of claim 1 wherein said at least one interbody device is substantially cylindrical in shape.

11. The method of claim 1 wherein said at least one interbody device is substantially rectangular or boxlike in shape.

12. The method of claim 1 where said cutting instruments prepare a space for accepting a substantially cylindrical implant.

13. The method of claim 1 where said cutting instruments prepare a space for accepting a substantially rectangular implant.

14. The method of claim 1 wherein said at least one cutting instrument, said at least one sizing instrument or said at least one interbody implant is used to distract said interbody space.

15. The method of claim 1 wherein said cutting instrument is a drill, awl or tap.

16. The method of claim 1 wherein said cutting instrument has external features for debris collection.

17. The method of claim 1 wherein said cutting instrument has at least one recess for debris collection.

18. The method of claim 1 wherein said interbody implant is a permanent implant for fusion or non-fusion purposes.

19. The method of claim 1 wherein placement of said at least one interbody implant further utilizes an insertion handle which utilizes said guide mechanism.

20. A device which is adapted for surgical placement over a guide mechanism for preparation of the interbody space having a self-distracting feature.

21. The device of claim 20 which has a feature such as a hole, slot or groove for mating with said guide mechanism.

22. The device of claim 20 which is substantially cylindrical.

23. The device of claim 20 which is non-cylindrical.

24. The device of claim 20 wherein the device has external threads and is screwed into said interbody space.

25. The device of claim 20 wherein the device is impacted into said interbody space.

26. The device of claim 20 wherein said device is a permanent implant.

27. The device of claim 20 wherein said device is used for the purpose of fusing said interbody space.

28. The device of claim 20 wherein said device is for non-fusion purposes.

29. The device of claim 20 having a tapered or rounded leading edge.

30. The device of claim 20 which is rotated to determine the interbody height.

31. The device of claim 20 having features to assist in disc removal or endplate preparation
